(19) United States
(12) Patent Application Publication
(10) Pub. No.: US 2014/0323920 A1
(43) Pub. Date: Oct. 30, 2014

(54) STABILIZED DEVICE FOR REMOTE PALPATION OF TISSUE IN TWO NON-COLLINEAR DIRECTIONS

(71) Applicant: Elwha LLC, Bellevue, WA (US)

(72) Inventors: Hon Wah Chin, Palo Alto, CA (US); Roderick A. Hyde, Redmond, WA (US); Jordin T. Kare, Seattle, WA (US); Elizabeth A. Sweeney, Seattle, WA (US); Lowell L. Wood, JR., Bellevue, WA (US)

(73) Assignee: Elwha LLC, a limited liability company of the State of Delaware

(21) Appl. No.: 13/873,489

(22) Filed: Apr. 30, 2013

(51) Int. Cl. A61B 5/00

(52) U.S. Cl. CPC A61B 5/0053
USPC 600/587

(57) ABSTRACT

Described embodiments include a handheld or hand operated device and a method. The method includes contacting a skin surface of a patient and palpating tissue in two non-collinear directions using a tip of a palpation element carried by a handheld or hand operated device. The palpation tip is movable with respect to the handheld or hand operated device in the two non-collinear directions. The method includes actively stabilizing the handheld or hand operated device in the two non-collinear directions during the palpating the tissue. The method includes characterizing a reaction of the tissue to the palpating in the two non-collinear directions. The method includes transmitting the characterization of the tissue reaction to a remote communication module.

### CROSS-REFERENCE TO RELATED APPLICATIONS

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

U.S. Pat. No. ______, entitled STABILIZED DEVICE FOR REMOTE PALPATION OF TISSUE, naming Hon Wah Chin, Roderick A. Hyde, Jordin T. Kare, Elizabeth A. Sweeney, and Lowell L. Wood, Jr. as inventors, filed 30 Apr. 2013 with attorney docket no. 0712-002-001-000000, is related to the present application.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

For example, and without limitation, an embodiment of the subject matter described herein includes a handheld or hand operated device. The device includes a palpation element having a tip configured to contact a skin surface of a patient and palpate tissue, and having a longitudinal axis. The device includes a handle structure configured to be held or gripped by a user operating the device. The device includes a body structure configured to allow (i) a palpation element tip range of travel relative to the body structure in two non-collinear directions, and (ii) a body structure range of travel relative to the handle structure in the two non-collinear directions. The device includes a palpation actuator coupling the body structure and the palpation element, and configured to reversibly move the palpation element relative to the body structure in the two non-collinear directions. The device includes a stabilization actuator coupling the body structure and the handle structure, and configured to reversibly move the body structure relative to the handle structure in the two non-collinear directions. The device includes a sensor configured to detect an acceleration of the body structure in the two non-collinear directions. The device includes a stabilization controller configured to stabilize the body structure by activating the stabilization actuator in a direction counteracting the detected acceleration of the body structure in the two non-collinear directions. The device includes a local communication module configured to communicate with a remote communication module. The device includes an examination controller configured to palpate the tissue underlying the skin surface with the tip of the palpation element in the two non-collinear directions by activating the palpation actuator in response to an instruction received from the remote communication module. The device includes a reporting circuit configured to characterize a reaction of the tissue or the skin surface to the palpation and to initiate a transmission of the characterization of the reaction by the local communication module to the remote communication module.

In an embodiment, the device includes an image capture module configured to acquire an image of the skin surface palpated by the palpation tip, and to initiate a transmission of the acquired image by the local communication module to the remote communication module. In an embodiment, the device includes a pointer module configured to indicate a selected portion of the skin surface of the patient to be palpated. In an embodiment, the device includes a touch probe module configured to conduct an ultrasound or resistivity measurement in conjunction with the palpation.

For example, and without limitation, an embodiment of the subject matter described herein includes a method. The method includes contacting a skin surface of a patient and palpating tissue in two non-collinear directions using a tip of a palpation element carried by a handheld or hand operated device. The palpation tip is movable with respect to the handheld or hand operated device in the two non-collinear directions. The method includes actively stabilizing the handheld or hand operated device in the two non-collinear directions during the palpating the tissue. The method includes characterizing a reaction of the tissue to the palpating in the two non-collinear directions. The method includes transmitting the characterization of the tissue reaction to a remote communication module. In an embodiment, the method includes receiving in the handheld or hand operated device an instruction to palpate the tissue in the two non-collinear directions from the remote communication module.

For example, and without limitation, an embodiment of the subject matter described herein includes a handheld or hand operated device. The device includes means for receiving an instruction from a remote communication module to palpate tissue underlying a portion of a skin of a patient in two non-collinear directions. The device includes means for palpating the tissue underlying the portion of the skin in response the received instruction, the palpating performed by a palpation means carried by the handheld or hand operated device and movable with respect to the handheld or hand operated device in the two non-collinear directions. The device includes means for stabilizing the handheld or hand operated device in the two non-collinear directions during the palpating the tissue. The device includes means for characterizing a reaction of the tissue to the palpating in two non-collinear directions. The device includes means for transmitting the characterization of the tissue reaction to the remote communication module. In an embodiment, the device includes means for transmitting a reaction force created by the stabilizing the handheld or hand operated device to a human user holding the handheld or hand operated device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example environment 200 in which embodiments may be implemented;

FIG. 2 illustrates additional details of the handheld or hand operated device 205 of FIG. 1;

FIG. 3 illustrates systems and electronics 250 of the handheld or hand operated device 205 of FIG. 1;

FIG. 4 illustrates an example operational flow 300;

FIG. 5 illustrates an embodiment of the operational flow 300 that may include at least one additional embodiment 360;

FIG. 6 illustrates a handheld or hand operated device 400;

FIG. 7 illustrates an example handheld or hand operated device 505;

FIG. 8 illustrates systems and electronics 550 of the handheld or hand operated device 505 of FIG. 7;

FIG. 9 illustrates an example operational flow 600; and

FIG. 10 illustrates an example handheld or hand operated device 1000.

### DETAILED DESCRIPTION

In the following detailed description, reference is made to the accompanying drawings, which form a part hereof. In the drawings, similar symbols typically identify similar components, unless context dictates otherwise. The illustrated embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various implementations by which processes and/or systems and/or other technologies described herein can be effected (e.g., hardware, software, and/or firmware), and that the preferred implementation will vary with the context in which the processes and/or systems and/or other technologies are deployed. For example, if an implementer determines that speed and accuracy are paramount, the implementer may opt for a mainly hardware and/or firmware implementation; alternatively, if flexibility is paramount, the implementer may opt for a mainly software implementation; or, yet again alternatively, the implementer may opt for some combination of hardware, software, and/or firmware. Hence, there are several possible implementations by which the processes and/or devices and/or other technologies described herein may be effected, none of which is inherently superior to the other in that any implementation to be utilized is a choice dependent upon the context in which the implementation will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to implement an operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described below. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, module, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Computing system environments typically includes a variety of computer-readable media products. Computer-readable media may include any media that can be accessed by a computing device and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by a computing device. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communications media may include wired media, such as a wired network and a direct-wired connection, and wireless media such as acoustic, RF, optical, and infrared media.

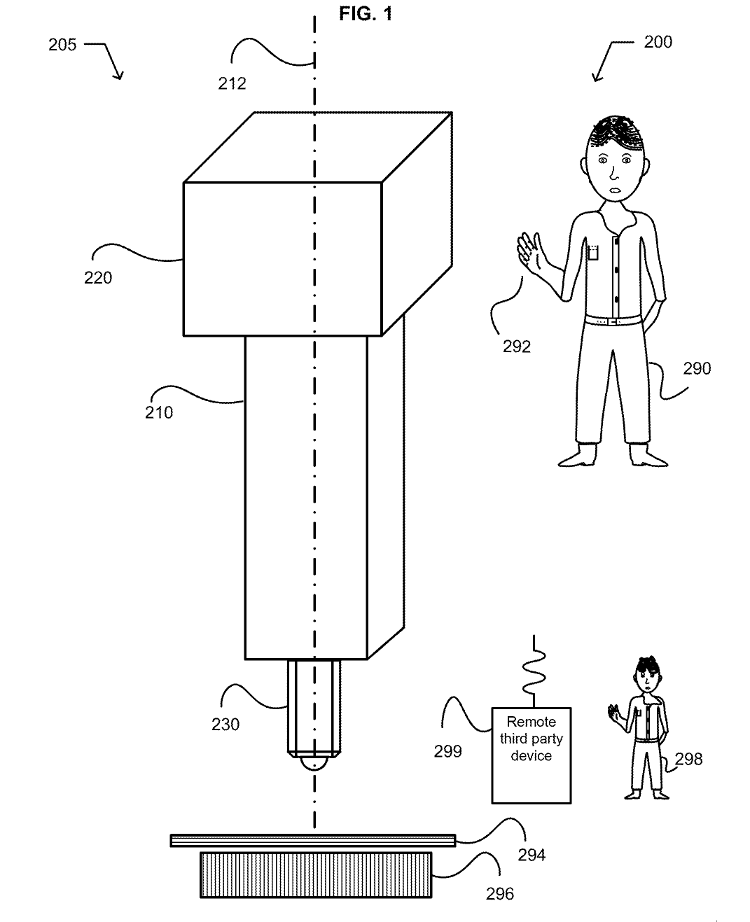

FIG. 1 illustrates an example environment 200 in which embodiments may be implemented. The environment includes a handheld or hand operated device 205, and a user 290 of the handheld or hand operated device. The user includes a hand 292 or other extremity suitable for the gripping or holding the handheld or hand operated device. The environment also includes a remote third-party 298, such as a health care provider, using a remote third-party device having a remote communication module 299 to communicate with the handheld or hand operated device. For example, the remote third-party device and the surgical instrument may communicate wirelessly, such as by Bluetooth or other wireless protocol, or may communicate over a network, such as a private network or a public network, such as the Internet. Also illustrated is a skin surface 294 of a patient and tissue 296 underlying or proximate to the skin surface.

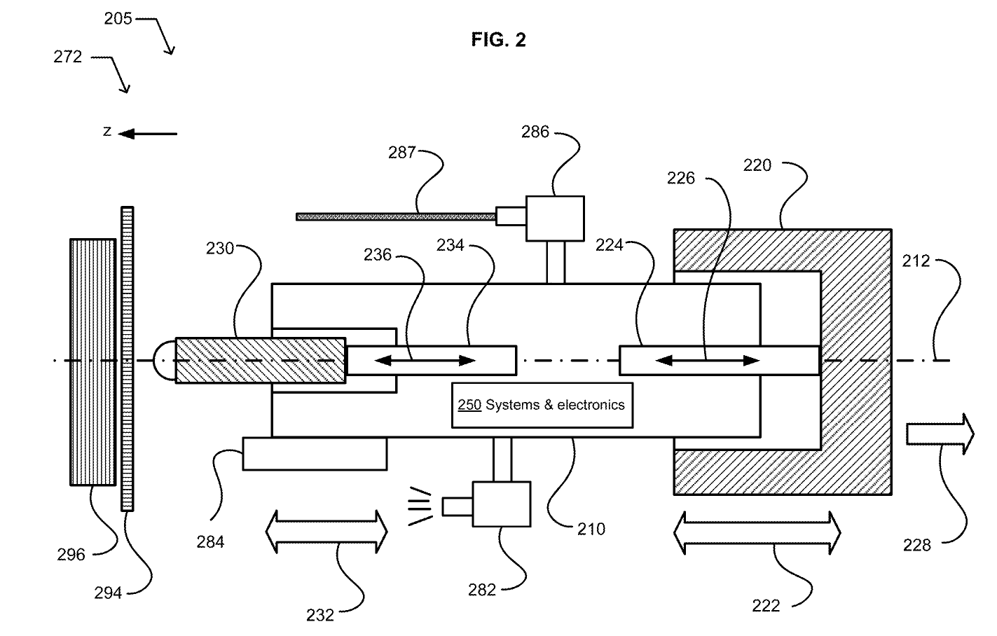

FIG. 2 illustrates additional details of the handheld or hand operated device 205 of FIG. 1. The handheld or hand operated device includes a palpation element 230 configured to palpate the tissue 296 underlying the skin surface 294 of the patient. The palpation element has a longitudinal axis 212, which is illustrated as along a Z axis 272. The handheld or hand operated device includes a handle structure 220 configured to be held or gripped by the user 290 operating the device. The handheld or hand operated device includes a body structure 210. The body structure is configured to allow a palpation element range of travel 232 relative to the body structure at least substantially parallel to the longitudinal axis. The body structure is configured to allow a body structure range of travel 222 relative to the handle structure at least substantially parallel to a longitudinal axis. The handheld or hand operated device includes a palpation linear actuator 234 coupling the body structure and the palpation element. The palpation linear actuator is configured to reversibly move 236 the palpation element relative to the body structure over at least a portion of the palpation element range of travel. For example, a palpation linear actuator may include an actuator that creates motion in a straight line. For example, a palpation linear actuator may be implemented using a mechanical, magnetic pneumatic, piezoelectric, or MEMS based actuator. For example, a palpation linear actuator may be implemented using a hydraulic or pressure actuator. In an embodiment, the palpation linear actuator includes a spring or lever actuator. In an embodiment, the examination controller 258 is configured to palpate the tissue with the palpation element by activating the palpation linear actuator in response to an instruction received from the remote communication module. In an embodiment, the examination controller is configured to palpate the tissue with the palpation element 230 by activating the palpation linear actuator in response to a predetermined sequence. In an embodiment, the examination controller is further configured to further palpate the tissue with the palpation element by activating the palpation linear actuator in response to the reaction of the tissue to the palpation.

The handheld or hand operated device includes a stabilization linear actuator 224 coupling the body structure and the handle structure. The stabilization linear actuator is configured to reversibly move 226 the body structure relative to the handle structure over at least a portion of the body structure range of travel.

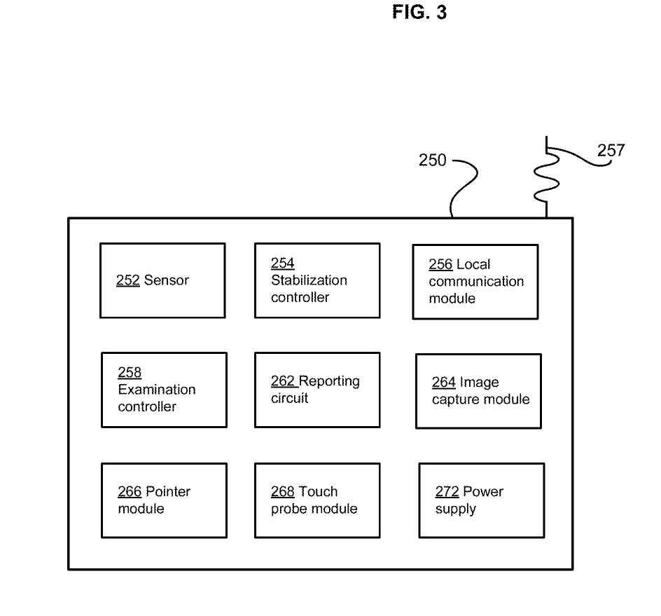

FIG. 3 illustrates systems and electronics 250 of the handheld or hand operated device 205 of FIG. 1. The systems and electronics include a sensor 252 configured to detect an acceleration of the body structure 210 along the longitudinal axis 212. The systems and electronics include a stabilization controller 254 configured to stabilize the body structure by activating the stabilization linear actuator 224 in a direction counteracting the detected acceleration of the body structure. The systems and electronics include a local communication module 256 configured to communicate with the remote communication module of the remote third-party device 299 illustrated in FIG. 1. The systems and electronics include an examination controller 258 configured to palpate the tissue 296 underlying the skin surface 294 with the palpation element 230 by activating the palpation linear actuator 234 in response to an instruction received from the remote communication module. The systems and electronics include a reporting circuit 262 configured to characterize a reaction of the tissue or the skin surface to the palpation, and to initiate a transmission of the characterization of the reaction by the local communication module to the remote communication module. For example, the initiated transmission may be a wireless transmission 257 or a wired transmission. In an embodiment, the systems and electronics include a power supply 272. In an embodiment, one or more elements of the systems and electronics may be located off board the body structure. For example, the power supply may be local to the body structure, and coupled with the other elements of the systems and electronics carried by the body structure.

In an embodiment, the tissue includes a soft tissue. For example, a tissue may include a connective tissue, a muscle tissue, an epithelial tissue, or a nervous tissue. For example, the tissue may include a mass, a tumor, a lymph node, a cyst (fluid-filled and/or solid). For example, the tissue may include a vascular tissue, an adipose tissue, or a cartilaginous tissue. In an embodiment, the tissue includes a skeletal tissue, for example a joint or a bone. In an embodiment, the tissue includes a dermal tissue. In an embodiment, the tissue includes an organ.

In an embodiment, palpation may be used by various therapists such as medical doctors, practitioners of chiropractic, osteopathic medicine, physical therapists, occupational therapists, and massage therapists as an assessment technique to examine the size, shape, consistency, texture, location, tenderness, and abnormality of an organ or body part. Assessment data that can be obtained through palpation includes identifying chest movement symmetry, skeletal abnormalities, skin abnormalities, pain, tenderness, swelling, circulation properties, and presence of masses. In an embodiment, palpation may be used to assess the texture of a patient's tissue (such as swelling or muscle tone), to locate the spatial coordinates of particular anatomical landmarks (e.g., to assess range and quality of joint motion or to assist in placement of medical equipment), and assess tenderness through tissue deformation (e.g. provoking pain with pressure or stretching). Palpation may be used either to determine painful areas and to qualify pain felt by patients, or to locate three-dimensional coordinates of anatomical landmarks to quantify some aspects of the palpated subject.

In an embodiment, the body structure 210 includes a portion located within an extension of the longitudinal axis 212. In an embodiment, the body structure includes a reaction mass (not illustrated). In an embodiment, the handle structure 220 is configured to be held or gripped by the patient or a third-party during a palpation of the tissue 296 of the patient. In an embodiment, the handle structure is further configured to transmit a reaction force 228 generated by the stabilization linear actuator 224 to the user operating the device. In an embodiment, the stabilization linear actuator includes a linear motor, linear piezomotor, extension motor, or multi-layer extension motor (stack actuator).

In an embodiment, the sensor 252 is further configured to detect a linear movement or an acceleration of the body structure 210 along the longitudinal axis 212. In an embodiment, the sensor includes a piezoelectric sensor. For example, the sensor may be integrated into the palpation linear actuator 234, as in a piezo patch. In an embodiment, the sensor includes a sensor internally referenced to the body structure. In an embodiment, the sensor includes an imaging device, for example a camera. For example, the camera may be configured to assist with stabilization by a reference to the skin. For example, the camera may take pictures of the patient and stabilize by maintaining the same view. In an embodiment, the sensor includes a MEMS sensor, which may incorporate an optical fiber. In an embodiment, the sensor includes an interferometric sensor. In an embodiment, the sensor includes an optical sensor. In an embodiment, the sensor includes an optical fiber sensor. For example, optical fiber strain sensors are described in Sylvie Delepine-Lesoille, et al., Optical fiber strain sensors for use in civil engineering, 272 BLPC 123 (October/November 2008). In an embodiment, the sensor includes an optical coherence tomography sensor. In an embodiment, the sensor includes an accelerometer. For example, the accelerometer may include a one to a four axis accelerometer. In an embodiment, the sensor includes a gyroscope, such as a MEMS gyroscope, a ring laser gyroscope, or an optical fiber gyroscope. For example, a MEMS gyroscope is marketed by Silicon Sensing System Japan as MEMS silicon ring gyro CRS03. (http://www.sssj.co.jp/en/products/gyro/crs03.html, accessed Apr. 29, 2013). For example, a ring laser and fiber optic gyroscope are described by Jeng-Nan Juang and R. Radharamanan, Evaluation of ring laser and fiber optic gyroscope technology, (https://docs.google.com/viewer?a=v&q=cache:PNuEILz6u -0J:www.asee.org/documents/sections/middle-atlantic/fall-2009/01-Evaluation-Of-Ring-Laser-And-Fiber-Optic-Gyroscope-Technology.pdf+&h1=en&gl=us&pid=bl&srcid=ADGEESjynJzDJ74kNLuHIHJmNU5k27p2u2Va0N15a_ugOS RkTlwEQC-zxAB6hA2HES7ZmgF4VR0EI-U28W4gONNda06dwGJZiCmAoAHI4-1f03UCBqw0siuSyzMyGLASeqQ4sl0cNk&sig=AHIEtbRenMriWLR3vfnmXPnpmubjXvL6Sw (accessed Apr. 2, 2013)).

In an embodiment, the stabilization controller 254 is configured to stabilize the body structure 210 with respect to the patient. In an embodiment, the stabilization controller includes a closed loop controller. In an embodiment, the closed loop controller includes a recursive filter. For example, a recursive filter may include a Kalman filter. For example, the recursive filter may be used in developing a stabilization response. In an embodiment, the stabilization controller is configured to stabilize the body structure with respect to an inertial reference frame. In an embodiment, the stabilization controller is configured to stabilize the body structure with respect to the skin surface of the patient. In an embodiment, the stabilization controller is configured to keep the body structure at rest during a palpation of the tissue underlying the skin surface. In an embodiment, the stabilization controller is configured to stabilize the body structure if the palpation linear actuator 234 is active.

In an embodiment, the palpation linear actuator 234 is configured to reversibly move the palpation element 230 relative to the body structure 210 if the stabilization controller 254 is active. In an embodiment, the palpation linear actuator is configured to reversibly move the palpation element relative to the body structure if (i) the stabilization controller is active and (ii) a criteria for stabilization of the body structure is met (e.g., body structure motion is below a threshold value). In an embodiment, the reporting circuit 262 is configured to characterize a reaction of the tissue 296 to the palpation as invalid if a criterion for stabilization of the body structure is not met.

In an embodiment, the palpation element 230 includes an examination element (not illustrated) configured to shear or pinch the skin surface 294 of the patient. In an embodiment, the palpation element includes a temperature sensor configured to measure a temperature of the contacted skin surface. In an embodiment, the reporting circuit 262 is further configured to initiate a report of the measured temperature of the contacted skin by the local communication module to the remote communication module. In an embodiment, the palpation linear actuator 234 is configured to deliver a palpation action by reversibly moving 236 the palpation element over at least a portion of the palpation element range of travel 232.

In an embodiment, the reporting circuit 262 is configured to characterize or quantify a reaction to the palpation by the tissue 296 of the patient. In an embodiment, the reporting circuit is configured to characterize a sonic reaction to the palpation of the tissue of the patient. In an embodiment, the reporting circuit is further configured to capture a sound resulting from the palpation of the tissue, and to initiate a transmission of the captured sound by the local communication module to the remote communication module. In an embodiment, the reporting circuit is configured to characterize a deformation reaction of the tissue or the skin surface to the palpation. In an embodiment, the reporting circuit is configured to characterize a firmness, color change, rebound, or a recovery to an original or normal state reaction of the tissue or the skin surface 294 to the palpation. In an embodiment, the reporting circuit is configured to characterize the palpation force applied to the tissue or the skin surface. In an embodiment, the reporting circuit is configured to characterize a reaction of the tissue or the skin surface to the palpation at least partially based on an interaction between the palpation element and the stabilized body structure. In an embodiment, the reporting circuit is configured to initiate a transmission by the local communication module 256 to the remote communication module of the third-party device 299. In an embodiment, the reporting circuit is further configured to process data indicative of the reaction of the tissue or the skin surface to the palpation. In an embodiment, the reporting circuit is further configured to acquire data indicative of the reaction of the tissue or the skin surface to the palpation. For example, the reporting circuit, the palpation element 230, or the palpation linear actuator 234 may include a pressure sensor or a force sensor configured to output data indicative of the reaction of the tissue or the skin surface to the palpation (e.g., the reaction force applied by the palpation element to the body structure). The transmission including (i) a description of the motion by the palpation element in palpating the tissue underlying the skin surface and (ii) the characterization of the reaction.

In an embodiment, the handheld or hand operated device 205 includes an imaging module 264 configured to acquire an image of the skin surface 294 palpated by the palpation element 230, and to initiate a transmission of the acquired image by the local communication module 256 to the remote communication module of the third-party device 299. In an embodiment, the imaging module includes an image capture device 282. In an embodiment, the image capture module is configured to acquire an image of the skin surface during a palpation by the palpation element. In an embodiment, the image capture module is further configured to acquire an image of the skin surface palpated by the palpation element in response to another instruction received from the remote communication module of the third-party device.

In an embodiment, the handheld or hand operated device 205 includes a pointer module 266 configured to indicate a selected portion of the skin surface 294 of the patient to be palpated. In an embodiment, the pointer module includes a pointing device 286. For example, the pointing device may include a steerable laser configured to direct a light beam 287 to indicate a selected portion of the skin surface of the patient to be palpated. In an embodiment, the pointer module is further configured to indicate a selected portion of the skin surface of the patient to be palpated in response to another instruction received from the remote communication module. In an embodiment, the pointer module is configured to indicate a region, such as by drawing a box or circle around a selected portion of the skin surface of the patient to be palpated. In an embodiment, the handheld or hand operated device includes a touch probe module 268 configured to conduct an ultrasound or resistivity measurement of the tissue in conjunction with the palpation. In an embodiment, the touch probe module includes a probe 284. In an embodiment, the touch probe module is configured to conduct an ultrasound or resistivity measurement in conjunction with the palpation in response to another instruction received from the remote communication module of the third-party device 299. In an embodiment, the handheld or hand operated device includes a touch probe module configured to conduct an acoustic measurement of the tissue in conjunction with the palpation. For example, an acoustic measurement may include an auscultatory, or percussive measurement.

In an embodiment, in use, the handheld or hand operated device 205 may generally be operated normal to the skin surface 294, for example with the longitudinal axis 212 generally normal to the skin surface.

In an embodiment, the handheld or hand operated device 205 may be used for remote examination of a patient in conjunction with the hand control unit and appurtenant devices described by U.S. Pat. No. 6,726,638, Direct examination of remote patient with virtual examination functionality, to Mark P. Ombrellaro (Apr. 27, 2004). In an embodiment, the handheld or hand operated device 205 may be used for remote examination of a patient in conjunction with the hand control unit and appurtenant devices described by U.S. Pat. No. 6,491,649, Device for the direct manual examination of a patient in a non-contiguous location, to Mark P. Ombrellaro (Dec. 10, 2002). In an embodiment, the handheld or hand operated device 205 may be used for remote examination of a patient in conjunction with the hand control unit and appurtenant devices described by U.S. Pat. App. Pub. No. 2004/0097836, Direct manual examination of a remove patient virtual examination functionality, to Mark P. Ombrellaro (May 20, 2004).

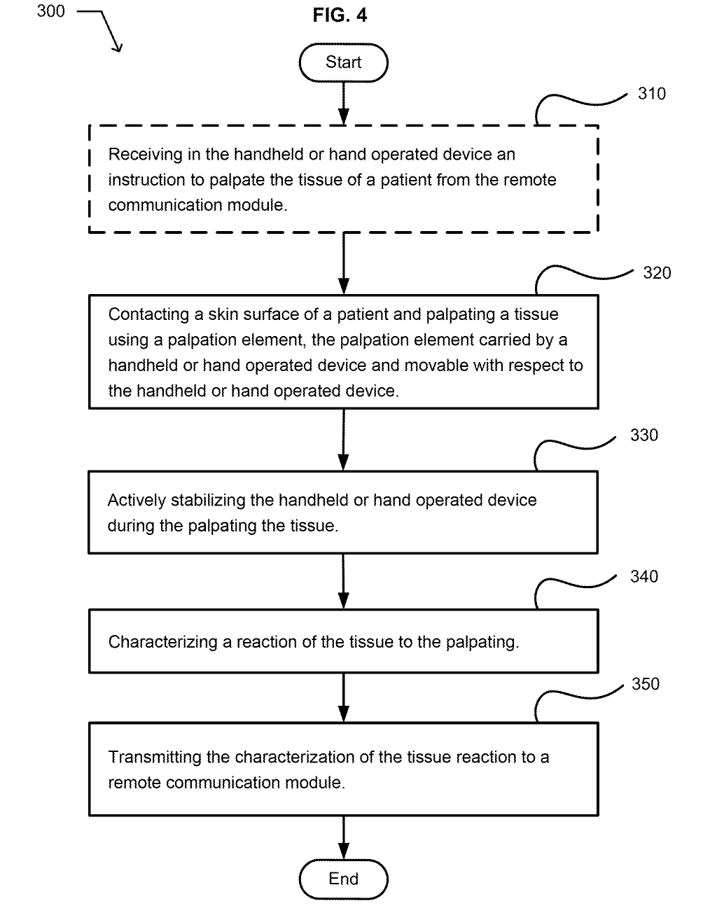

FIG. 4 illustrates an example operational flow 300. After a start operation, the operation flow includes an examination operation 320. The examination operation includes contacting a skin surface of a patient and palpating a tissue using a palpation element. The palpation element carried by a handheld or hand operated device and movable with respect to the handheld or hand operated device. In an embodiment, the examination operation may be implemented using the palpation element 230, the palpation linear actuator 234, and the examination controller 258 as described in conjunction with FIGS. 1-3. A steadying operation 330 includes actively stabilizing the handheld or hand operated device during the palpating the tissue. In an embodiment, the steadying operation may be implemented using the handle structure 220, the stabilization linear actuator 224, and the stabilization controller 254 to steady the body structure 210 as described in conjunction with FIGS. 1-3. A description operation 340 includes characterizing a reaction of the tissue to the palpating. In an embodiment, the description operation may be implemented using the reporting circuit 262 described in conjunction with FIGS. 1-3. A communication operation 350 includes transmitting the characterization of the tissue reaction to a remote communication module. In an embodiment, the communication operation may be implemented using the reporting circuit 262 to communicate with the remote communication module of the third-party device 299 as described in conjunction with FIGS. 1-3. The operational flow includes an end operation.

In an embodiment, the operational flow 300 includes a reception operation 310. The reception operation includes receiving in the handheld or hand operated device an instruction to palpate the tissue of a patient from the remote communication module. In an embodiment, the reception operation may be implemented using the local communication module 256 of the handheld or hand operated device 205 to receive the instruction from the remote communication module of the third-party device 299 as described in conjunction with FIGS. 1-3. In an embodiment, the instruction includes an instruction to palpate tissue of a patient proximate to or touching a contact region of the device. In an embodiment, the operational flow includes initiating the palpating the issue after receiving an indication of the stabilizing the handheld or hand operated device.

In an embodiment of the examination operation 320, the palpating tissue includes palpating tissue of a patient in response to an indication of the stabilizing the handheld or hand operated device. In an embodiment of the examination operation, the palpating tissue includes palpating tissue of a patient in response to a predetermined sequence. In an embodiment of the examination operation, the palpating tissue further includes another palpating tissue of a patient in response to the characterized reaction of the tissue to palpation. For example, a palpation sequence may be adjusted based on results of a previous palpitation. In an embodiment of the examination operation, the palpating tissue includes palpating tissue in a direction having a component perpendicular to the skin surface of the patient. In an embodiment of the examination operation, the palpating tissue includes palpating tissue in a direction having a component parallel to the skin surface of the patient.

In an embodiment of the steadying operation 330, the stabilizing includes actively stabilizing the handheld or hand operated device with respect to the patient during the palpating the tissue.

In an embodiment, the reception operation 310 includes receiving in a handheld or hand operated device an instruction from a remote communication module to palpate tissue underlying a portion of a skin of a patient proximate to or touching a contact region of the device. In an embodiment, the steadying operation 330 includes stabilizing the handheld or hand operated device with respect to the patient during the palpating the tissue. In an embodiment, the steadying operation includes actively stabilizing the handheld or hand operated device with respect to an inertial reference frame during the palpating the tissue. In an embodiment, the actively steadying operation includes stabilizing the handheld or hand operated device with respect to the portion of the skin during the palpating the tissue. In an embodiment, the communication operation 350 includes transmitting (i) a description of the motion of the palpation element in palpating the tissue underlying the skin surface and (ii) the characterization of the reaction.

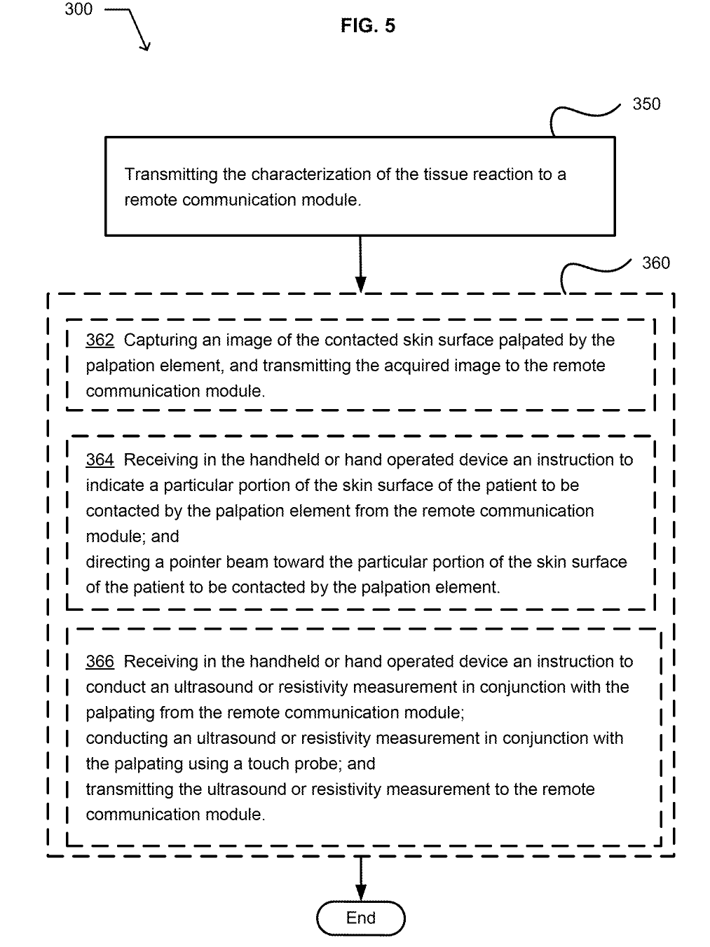

FIG. 5 illustrates an embodiment of the operational flow 300 that may include at least one additional embodiment 360. The at least one additional embodiment may include an operation 362, an operation 364, or an operation 366. The operation 362 includes capturing an image of the contacted skin surface palpated by the palpation element, and transmitting the acquired image to the remote communication module. The operation 364 includes receiving in the handheld or hand operated device an instruction to indicate a particular portion of the skin surface of the patient to be palpated from the remote communication module. The operation 364 also includes directing a pointer beam toward the particular portion of the skin surface of the patient to be contacted by the palpation element. The operation 366 includes receiving in the handheld or hand operated device an instruction to conduct an ultrasound or resistivity measurement in conjunction with the palpating from the remote communication module. The operation 366 also includes conducting an ultrasound or resistivity measurement in conjunction with the palpating using a touch probe. The operation 366 further includes transmitting the ultrasound or resistivity measurement to the remote communication module.

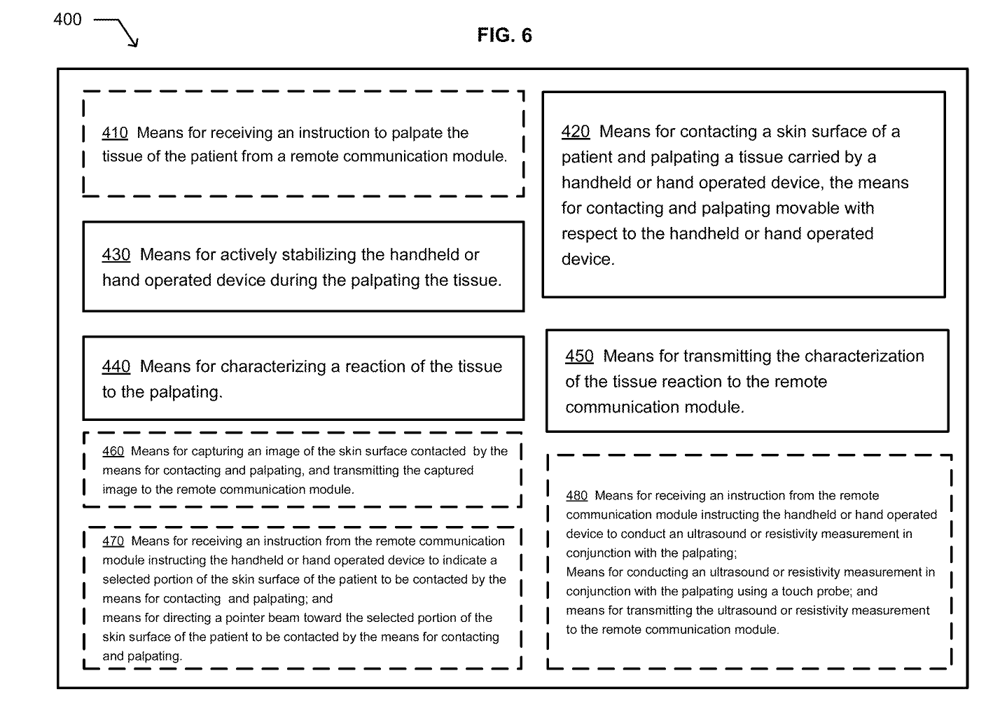

FIG. 6 illustrates a handheld or hand operated device 400. The device includes means 420 contacting a skin surface of a patient and palpating a tissue carried by a handheld or hand operated device, the means for contacting and palpating is movable with respect to the handheld or hand operated device. The device includes means 430 for actively stabilizing the handheld or hand operated device during the palpating the tissue. The device includes means 440 for characterizing a reaction of the tissue to the palpating. The device includes means 450 for transmitting the characterization of the tissue reaction to the remote communication module.

In an embodiment, the device 400 includes means 410 for receiving an instruction to palpate the tissue of the patient from a remote communication module. In an embodiment, the device includes means 460 for capturing an image of the skin surface contacted by the means for contacting and palpating, and transmitting the captured image to the remote communication module. In an alternative embodiment, the device includes means 470 for receiving an instruction from the remote communication module instructing the handheld or hand operated device to indicate a selected portion of the skin surface of the patient to be palpated. The means 470 also includes means for directing a pointer beam toward the selected portion of the skin surface of the patient to be contacted by the means for contacting and palpating. In an alternative embodiment, the device includes means 480 for receiving an instruction from the remote communication module instructing the handheld or hand operated device to conduct an ultrasound or resistivity measurement in conjunction with the palpating. The means 480 also includes means for conducting an ultrasound or resistivity measurement in conjunction with the palpating using a touch probe. The means 480 further includes means for transmitting the ultrasound or resistivity measurement to the remote communication module.

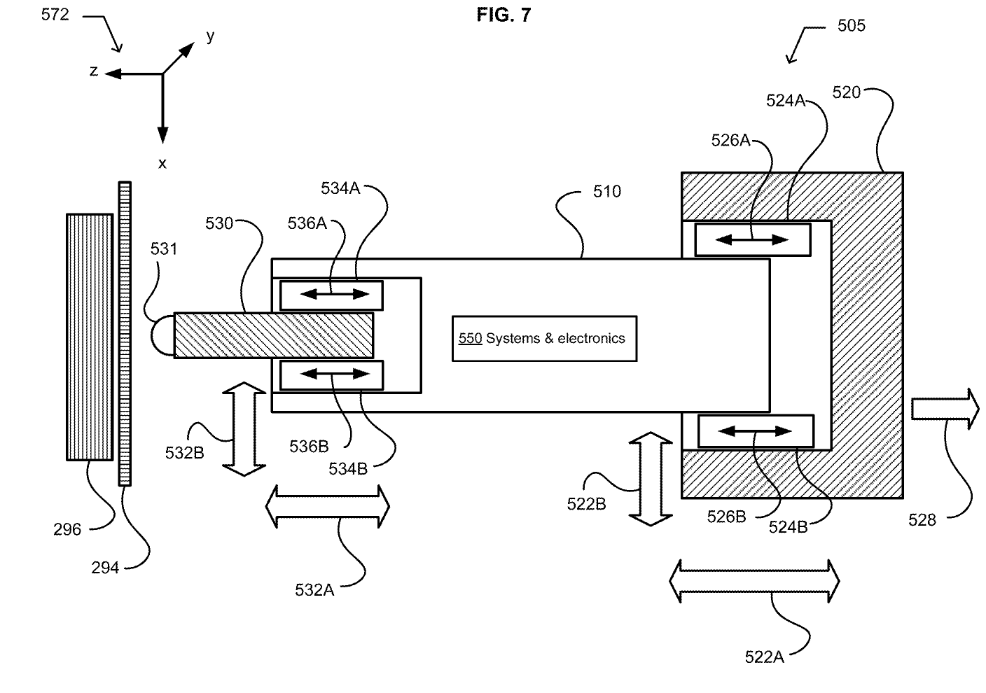

FIG. 7 illustrates an example handheld or hand operated device 505. The handheld or hand operated device includes a palpation element 530 having a tip 531 configured to contact a skin surface 294 of a patient and palpate a tissue 296. The handheld or hand operated device includes a handle structure 520 configured to be held or gripped by a user operating the device. For example, the user may include the user 292 described in conjunction with FIG. 1. The handheld or hand operated device includes a body structure 510. The body structure is configured to allow a palpation element tip range of travel relative to the body structure in two non-collinear directions, which are illustrated as palpation element tip range of travel 532A and palpation element tip range of travel 532B. The two non-collinear directions may be referenced relative to the X, Y, and Z axis of a coordinate system 572, or alternatively any other coordinate system. In an embodiment, the coordinate system may be a Cartesian coordinate system. The body structure is configured to allow a body structure range of travel relative to the handle structure in the two non-collinear directions, which are illustrated as body structure range of travel 522A and body structure range of travel 522B.

The handheld or hand operated device 505 includes a palpation actuator 534 coupling the body structure and the palpation element. In an embodiment illustrated in FIG. 7, the palpation actuator includes a first palpation actuator 534A and a second palpation actuator 534B. The palpation actuator is configured to reversibly move the palpation element relative to the body structure in the two non-collinear directions, which are respectively illustrated as reversible movement 536A and reversible movement 536B. For example, activating the first palpation actuator and the second palpation actuator in concert will move the tip along the Z axis toward and away from the skin surface. For example, activating the first palpation actuator and the second palpation actuator in opposition will tilt the palpation element about the Y axis, thereby moving the tip along the X axis stroking the skin surface. The handheld or hand operated device includes a stabilization actuator 524 coupling the body structure 510 and the handle structure 520. In an embodiment illustrated in FIG. 7, the stabilization actuator includes a first stabilization actuator 524A and a second stabilization actuator 524B. The stabilization actuator is configured to reversibly move the body structure relative to the handle structure in the two non-collinear directions. For example, activating the first stabilization actuator and the second stabilization actuator in concert will move the body structure along the Z axis toward and away from the handle structure. For example, activating the first stabilization actuator and the second stabilization actuator in opposition will move the hand structure along the X axis by tilting it about the Y axis with respect to the body structure. In another embodiment (not shown) palpation actuators 534A and 534B act directly along the Z and X axes respectively in order to move the palpation element with reversible movement 536A along the Z axis and with reversible movement 536B along the X axis. Similarly, in another embodiment (not shown) stabilization actuators 524A and 524B act directly along the Z and X axes respectively in order to move the body element with reversible movement 526A along the Z axis and with reversible movement 526B along the X axis. In other embodiments, different configurations of the palpation actuators 534 and the stabilization actuators 524 can be employed; for instance, X motion 532B may be induced by rotation of the palpation element about the Y axis (as above), but this might be responsive to a rotational actuator rather than two opposing linear actuators. In another potential variation, stabilization actuator 524 may utilize three or more separate actuators (rather than two) to induce the non-collinear motions 522A and 522B. It will be appreciated that a designer may select from many different actuator configurations in order to achieve the non-collinear motions of the palpation element relative to the body structure and relative to the handle structure. It will also be appreciated that the two non-collinear directions may be along Y and Z axes, may be along X and Y axes, may be along axes different than X, Y, or Z, may be along non-orthogonal axes, etc.

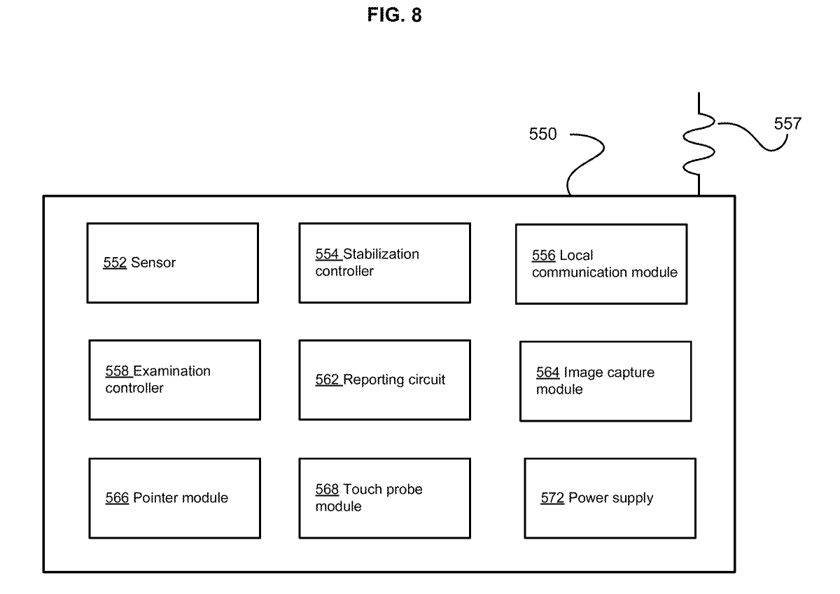

FIG. 8 illustrates systems and electronics 550 of the handheld or hand operated device 505 of FIG. 7. The systems and electronics include a sensor 552 configured to detect an acceleration of the body structure 510 in the two non-collinear directions. The systems and electronics include a stabilization controller 554 configured to stabilize the body structure 510 by activating the stabilization actuator 524 in a direction counteracting the detected acceleration of the body structure in the two non-collinear directions. The systems and electronics include a local communication module 556 configured to communicate with a remote communication module, such as the remote communication module of the remote third-party device 299 described in conjunction with FIG. 1. The systems and electronics include an examination controller 558 configured to palpate the tissue 296 with the tip 531 of the palpation element 530 in the two non-collinear directions by activating the palpation actuator 534. The systems and electronics include a reporting circuit 562 configured to characterize a reaction of the tissue or the skin surface to the palpation, and to initiate a transmission of the characterization of the reaction by the local communication module to the remote communication module. For example, the initiated transmission may be a wireless transmission 557 or a wired transmission. In an embodiment, the systems and electronics include a power supply 572. In an embodiment, one or more elements of the systems and electronics may be located off board the body structure. For example, the power supply may be local to the body structure, and coupled with the other elements of the systems and electronics carried by the body structure.

Continuing with FIGS. 7 and 8, in an embodiment of the handheld or hand operated device 505, the handle structure 510 is further configured to transmit a received force 528 from the stabilization actuator 524 to the user operating the handheld or hand operated device, such as the user 290 described in conjunction with FIG. 1. In an embodiment, the palpation element tip 531 is configured to palpate tissue and to pressure or stroke tissue. In an embodiment, the body structure 510 is configured to allow a palpation element tip range of travel relative to the body structure in two orthogonal directions. For example, the Z and X axis of the coordinate system 572 may be orthogonal directions. In an embodiment, the body structure is configured to allow a palpation element tip range of travel relative to the body structure in three non-collinear directions. For example, the X, Y, and Z axis of the coordinate system may be three non-collinear directions. In an embodiment, the body structure is configured to allow a palpation element tip range of travel relative to the body structure in three orthogonal directions. For example, the X, Y, and Z axis of the coordinate system may be orthogonal.

In an embodiment of the handheld or hand operated device 505, the palpation actuator 534 is configured to reversibly move 536 the palpation tip 531 relative to the body structure 510 in the two non-collinear directions over at least a portion of the palpation element range of travel 532. In an embodiment, the palpation actuator includes a linear actuator or a bending actuator. For example, a bending actuator may include a Piezo Bender Actuator manufactured by PI (Physik Instrumente) of Karlsruhe, Germany. In an embodiment, the bending actuator includes a piezoelectric bending actuator. For example, the bending actuator may include a micro piezoelectric bending actuator. In an embodiment, the bending actuator includes a piezoelectric strip actuator. In an embodiment, the bending actuator includes a piezoelectric bimorph actuator. In an embodiment, the bending actuator includes a piezoelectric multimorph actuator. In an embodiment, the bending actuator includes a piezoelectric patch actuator. In an embodiment, the bending actuator includes a magnetostricitive actuator. In an embodiment, the bending actuator includes a shape memory actuator. In an embodiment, the palpation actuator includes a flexible beam element having at least two actuators configured to reversibly bend the flexible beam element with respect to the two non-collinear directions. In an embodiment, the palpation actuator is activated in response to an instruction received from the remote communication module. In an embodiment, the palpation actuator is activated in response to a predetermined sequence. In an embodiment, the palpation actuator is activated in response to the reaction of the tissue to the palpation.

In an embodiment of the handheld or hand operated device 505, the stabilization actuator 524 is configured to reversibly move 526 the body structure relative to the handle structure in the two non-collinear directions over at least a portion of the body structure range of travel 522. In an embodiment, the stabilization actuator includes a linear actuator or a bending actuator. In an embodiment, the stabilization actuator includes a flexible beam element having at least two actuators configured to reversibly bend the flexible beam element with respect to the two non-collinear directions.

In an embodiment, the handheld or hand operated device 505 includes an image capture module 564 configured to acquire an image of the skin surface contacted by the palpation tip 531, and to initiate a transmission of the acquired image by the local communication module 556 to the remote communication module of the third-party remote device 299. In an embodiment, the image capture module may include the image capture device 282 described in conjunction with FIG. 2. In an embodiment, the handheld or hand operated device includes a pointer module 566 configured to indicate a selected portion of the skin surface of the patient to be contacted by the palpation tip. In an embodiment, the pointer module includes the pointing device 286 described in conjunction with FIG. 2. In an embodiment, the handheld or hand operated device includes a touch probe module 568 configured to conduct an ultrasound or resistivity measurement in conjunction with the palpation. In an embodiment, the touch probe module the probe 284 described in conjunction with FIG. 2.

In an embodiment, the handheld or hand operated device 505 may be used for remote examination of a patient in conjunction with the hand control unit and appurtenant devices described by U.S. Pat. No. 6,726,638, Direct examination of remote patient with virtual examination functionality, to Mark P. Ombrellaro (Apr. 27, 2004). In an embodiment, the handheld or hand operated device may be used for remote examination of a patient in conjunction with the hand control unit and appurtenant devices described by U.S. Pat. No. 6,491,649, Device for the direct manual examination of a patient in a non-contiguous location, to Mark P. Ombrellaro (Dec. 10, 2002). In an embodiment, the handheld or hand operated device may be used for remote examination of a patient in conjunction with the hand control unit and appurtenant devices described by U.S. Pat. App. Pub. No. 2004/0097836, Direct manual examination of a remove patient virtual examination functionality, to Mark P. Ombrellaro (May 20, 2004).

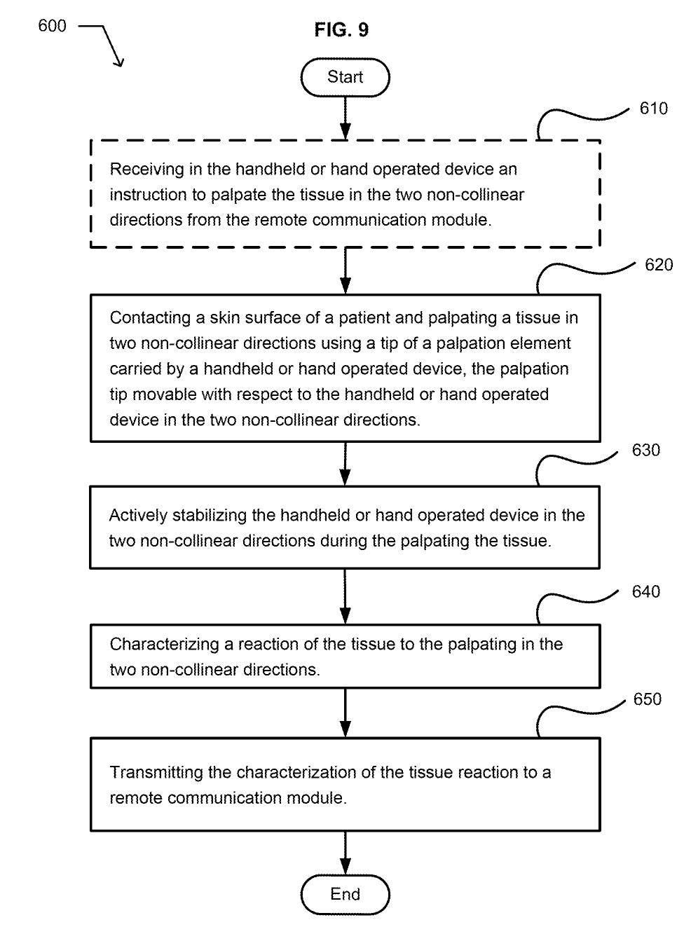

FIG. 9 illustrates an example operational flow 600. After a start operation, the operation flow includes an examination operation 620. The examination operation 620 includes contacting a skin surface of a patient and palpating a tissue in two non-collinear directions using a tip of a palpation element carried by a handheld or hand operated device. The palpation tip is movable with respect to the handheld or hand operated device in the two non-collinear directions. In an embodiment, the examination operation may be implemented using the palpation element 530, the palpation tip 531, the palpation actuator 234, and the examination controller 558 as described in conjunction with FIGS. 7-8. A steadying operation 630 includes actively stabilizing the handheld or hand operated device in the two non-collinear directions during the palpating the tissue. In an embodiment, the steadying operation may be implemented using the handle structure 520, the stabilization actuator 524, and the stabilization controller 554 to steady the body structure 510 as described in conjunction with FIGS. 7-8. A description operation 640 includes characterizing a reaction of the tissue to the palpating in the two non-collinear directions. In an embodiment, the description operation may be implemented using the reporting circuit 562 described in conjunction with FIGS. 7-8. A communication operation 650 includes transmitting the characterization of the tissue reaction to a remote communication module. In an embodiment, the communication operation may be implemented using the reporting circuit 562 to communicate with the remote communication module of the third-party device 299 as described in conjunction with FIGS. 7-8. The operational flow includes an end operation.

In an embodiment, the steadying operation 630 includes actively stabilizing the handheld or hand operated device in the two non-collinear directions with respect to the patient during the palpating the tissue. In an embodiment, the steadying operation includes actively stabilizing the handheld or hand operated device in the two non-collinear directions with respect to an inertial reference frame during the palpating the tissue. In an embodiment, the steadying operation includes actively stabilizing the device in the two non-collinear directions with respect to the portion of the skin during the palpating the tissue. In an embodiment, the communication operation 650 includes transmitting (i) a description of the motion of the palpation element in palpating the tissue underlying the skin surface in the two non-collinear directions and (ii) the characterization of the reaction. In an embodiment, the operational flow includes an operation transmitting a reaction force created by the stabilizing the handheld or hand operated device to a human user holding the handheld or hand operated device.

In an embodiment, the operational flow 900 includes a reception operation 910. The reception operation includes receiving in the handheld or hand operated device an instruction to palpate the tissue in the two non-collinear directions from the remote communication module. In an embodiment, the reception operation may be implemented using the local communication module 556 of the handheld or hand operated device 505 to receive the instruction from the remote communication module of the third-party device 299 as described in conjunction with FIGS. 7-8. In an embodiment, the operational flow includes initiating the contacting and palpating.

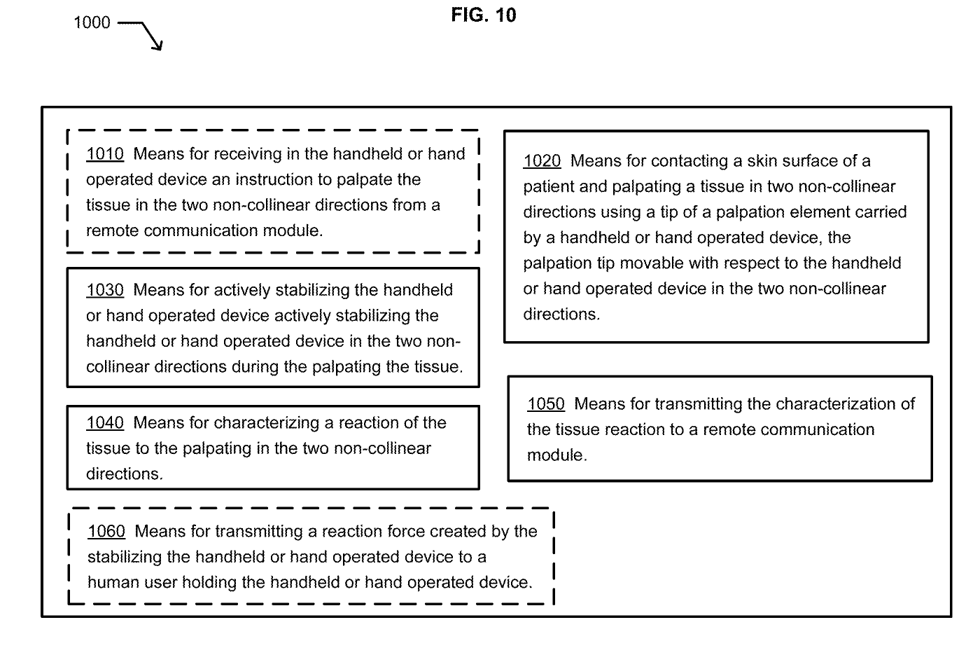

FIG. 10 illustrates an example handheld or hand operated device 1000. The device includes means 1020 for contacting a skin surface of a patient and palpating a tissue in two non-collinear directions using a tip of a palpation element. The palpation tip is carried by a handheld or hand operated device and movable with respect to the handheld or hand operated device in the two non-collinear directions. The device includes means 1030 for actively stabilizing the handheld or hand operated device in the two non-collinear directions during the palpating the tissue. The device includes means 1040 for characterizing a reaction of the tissue to the palpating in two non-collinear directions. The device includes means 1050 for transmitting the characterization of the tissue reaction to a remote communication module. In an embodiment, the device includes means 1060 for transmitting a reaction force created by the stabilizing the handheld or hand operated device to a human user holding the handheld or hand operated device.

In an embodiment, the device 1000 includes means 1010 for receiving in the handheld or hand operated device an instruction from the remote communication module to palpate the tissue in the two non-collinear directions. In an embodiment, the device includes means for transmitting a reaction force created by the stabilizing the handheld or hand operated device to a human user holding the handheld or hand operated device.

All references cited herein are hereby incorporated by reference in their entirety or to the extent their subject matter is not otherwise inconsistent herewith.

In some embodiments, “configured” includes at least one of designed, set up, shaped, implemented, constructed, or adapted for at least one of a particular purpose, application, or function.

It will be understood that, in general, terms used herein, and especially in the appended claims, are generally intended as “open” terms. For example, the term “including” should be interpreted as “including but not limited to.” For example, the term “having” should be interpreted as “having at least.” For example, the term “has” should be interpreted as “having at least.” For example, the term “includes” should be interpreted as “includes but is not limited to,” etc. It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

In those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Use of “Start,” “End,” “Stop,” or the like blocks in the block diagrams is not intended to indicate a limitation on the beginning or end of any operations or functions in the diagram. Such flowcharts or diagrams may be incorporated into other flowcharts or diagrams where additional functions are performed before or after the functions shown in the diagrams of this application. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A handheld or hand operated device comprising:
a palpation element having a tip configured to contact a skin surface of a patient and palpate tissue underlying or proximate to the skin surface;
a handle structure configured to be held or gripped by a user operating the device;
a body structure configured to allow (i) a palpation element tip range of travel relative to the body structure in two non-collinear directions, and (ii) a body structure range of travel relative to the handle structure in the two non-collinear directions;
a palpation actuator coupling the body structure and the palpation element, and configured to reversibly move the palpation element relative to the body structure in the two non-collinear directions;
a stabilization actuator coupling the body structure and the handle structure, and configured to reversibly move the body structure relative to the handle structure in the two non-collinear directions;
a sensor configured to detect an acceleration of the body structure in the two non-collinear directions;
a stabilization controller configured to stabilize the body structure by activating the stabilization actuator in a direction counteracting the detected acceleration of the body structure in the two non-collinear directions;
a local communication module configured to communicate with a remote communication module;
an examination controller configured to palpate the tissue with the tip of the palpation element in the two non-collinear directions by activating the palpation actuator; and
a reporting circuit configured to characterize a reaction of the tissue to the palpation, and to initiate a transmission of the characterization of the reaction by the local communication module to the remote communication module.

2. The device of claim 1, wherein the handle structure is further configured to transmit a received force from the stabilization actuator to the user operating the device.

3. The device of claim 1, wherein the palpation element tip is configured to palpate tissue and to pressure or stroke the tissue of the patient.

4. The device of claim 1, wherein the body structure is configured to allow a palpation element tip range of travel relative to the body structure in two orthogonal directions.

5. The device of claim 1, wherein the body structure is configured to allow a palpation element tip range of travel relative to the body structure in three non-collinear directions.

6. The device of claim 1, wherein the body structure is configured to allow a palpation element tip range of travel relative to the body structure in three orthogonal directions.

7. The device of claim 1, wherein the palpation actuator is configured to reversibly move the palpation tip relative to the body structure in the two non-collinear directions over at least a portion of the palpation element range of travel.

8. The device of claim 1, wherein the palpation actuator includes a linear actuator or a bending actuator.

9. The device of claim 1, wherein the palpation actuator includes a flexible beam element having at least two actuators configured to reversibly bend the flexible beam element with respect to the two non-collinear directions.

10. The device of claim 1, wherein the palpation actuator is activated in response to an instruction received from the remote communication module.

11. The device of claim 1, wherein the palpation actuator is activated in response to a predetermined sequence.

12. The device of claim 1, wherein the palpation actuator is activated in response to the reaction of the tissue to the palpation.

13. The device of claim 1, wherein the stabilization actuator is configured to reversibly move the body structure relative to the handle structure in the two non-collinear directions over at least a portion of the body structure range of travel.

14. The device of claim 1, wherein the stabilization actuator includes a linear actuator or a bending actuator.

15. The device of claim 1, wherein the stabilization actuator includes a flexible beam element having at least two actuators configured to reversibly bend the flexible beam element with respect to the two non-collinear directions.

16. The device of claim 1, further comprising:
an image capture module configured to acquire an image of the skin surface contacted by the palpation tip, and to initiate a transmission of the acquired image by the local communication module to the remote communication module.

17. The device of claim 1, further comprising:
a pointer module configured to indicate a selected portion of the skin surface of the patient to be contacted by the palpation tip.

18. The device of claim 1, further comprising:
a touch probe module configured to conduct an ultrasound or resistivity measurement in conjunction with the palpation.

19. A method comprising:
contacting a skin surface of a patient using a tip of a palpation element carried by a handheld or hand operated device, the palpation tip movable with respect to the handheld or hand operated device in two non-collinear directions;
palpating in the two non-collinear directions a tissue underlying or proximate to the skin surface using a palpation element;
actively stabilizing the handheld or hand operated device in the two non-collinear directions during the palpating the tissue;
characterizing a reaction of the tissue to the palpating in the two non-collinear directions; and
transmitting the characterization of the tissue reaction to a remote communication module.

20. The method of claim 19, wherein the actively stabilizing includes actively stabilizing the handheld or hand operated device in the two non-collinear directions with respect to the patient during the palpating the tissue.

21. The method of claim 19, wherein the actively stabilizing includes actively stabilizing the handheld or hand operated device in the two non-collinear directions with respect to an inertial reference frame during the palpating the tissue.

22. The method of claim 19, wherein the actively stabilizing includes actively stabilizing the device in the two non-collinear directions with respect to the skin surface during the palpating the tissue.

23. The method of claim 19, wherein the transmitting includes transmitting (i) a description of the motion of the palpation element in palpating the tissue in the two non-collinear directions and (ii) the characterization of the reaction.

24. The method of claim 19, further comprising:
receiving in the handheld or hand operated device an instruction to palpate the tissue in the two non-collinear directions from the remote communication module.

25. The method of claim 19, further comprising:
initiating the palpating after receiving an indication of the stabilizing the handheld or hand operated device.

26. The method of claim 19, further comprising:
transmitting a reaction force created by the stabilizing the handheld or hand operated device to a human user holding the handheld or hand operated device.

27. A handheld or hand operated device comprising:
means for contacting a skin surface of a patient using a tip of a palpation element carried by a handheld or hand operated device, the palpation tip movable with respect to the handheld or hand operated device in two non-collinear directions;
means for palpating in the two non-collinear directions a tissue underlying or proximate to the skin surface using a palpation element;
means for actively stabilizing the handheld or hand operated device in the two non-collinear directions during a palpation of the tissue by the means for palpating;
means for characterizing a reaction of the tissue to the palpating in the two non-collinear directions; and
means for transmitting the characterization of the tissue reaction to a remote communication module.

28. The device of claim 27, further comprising:
means for receiving in the handheld or hand operated device an instruction from the remote communication module to palpate the tissue in the two non-collinear directions.

29. The device of claim 27, further comprising:
means for transmitting a reaction force created by the stabilizing the handheld or hand operated device to a human user holding the handheld or hand operated device.
